(19) United States
(12) Patent Application Publication
(10) Pub. No.: US 2018/0236089 A1
(43) Pub. Date: Aug. 23, 2018

(54) RADIATION EMITTING PEPTIDE NUCLEIC ACID CONJUGATES AND USES THEREOF FOR DIAGNOSIS, IMAGING, AND TREATMENT OF DISEASES, CONDITIONS AND DISORDERS

(71) Applicant: Yissum Research Development Company of the Hebrew University of Jerusalem Ltd., Jerusalem (IL)

(72) Inventors: Eylon YAVIN, Jerusalem (IL); Abraham RUBINSTEIN, Jerusalem (IL); Netanel KOLEVZON, Jerusalem (IL)

(73) Assignee: Yissum Research Development Company of the Hebrew University of Jerusalem Ltd., Jerusalem (IL)

(21) Appl. No.: 15/751,654

(22) PCT Filed: Aug. 11, 2016
(86) PCT No.: PCT/IL2016/050878

§ 371 Date: Feb. 9, 2018

Related U.S. Application Data
(60) Provisional application No. 62/203,526, filed on Aug. 11, 2015.

(51) Int. Cl. A61K 47/54; A61K 47/64; A61K 47/65; A61K 48/00; C07K 7/08; C12N 15/113; A61K 49/00

(52) U.S. Cl. CPC A61K 47/549; A61K 47/64; A61K 47/65; A61K 49/0056; C07K 7/08; C12N 15/1138; A61K 49/0032; A61K 48/0041

(57) ABSTRACT

Provided is a conjugate including at least one radiation emitting probe and at least one gene complementary component for use in highly sensitive diagnosis, imaging, and treatment of conditions, diseases and disorders, including compositions including said conjugates and kits thereof.

### TECHNOLOGICAL FIELD

The present invention provides radiation emitting (red to NIR) PNA conjugates for highly sensitive diagnosis, imaging, and treatment of conditions, diseases and disorders.

### BACKGROUND ART

[1] Maebert, K., Cojoc, M., Peitzsch, C., Kurth, I., Souchelnytskyi, S., and Dubrovska, A. (2014) Cancer biomarker discovery: Current status and future perspectives, Int. J. Radiation Biol. 90, 659-677.
[2] Brink, M., De Goeij, A. F. P. M., Weijenberg, M. P., Roemen, G. M. J. M., Lentjes, M. H. F. M., Pachen, M. M. M., Smits, K. M., De Bruine, A. P., Goldbohm, R. A., and Van Den Brandt, P. A. (2003) K-ras oncogene mutations in sporadic colorectal cancer in The Netherlands Cohort Study, Carcinogenesis 24, 703-710.
[3] Heinzerling, L., Baiter, M., Kuehnapfel, S., Schuler, G., Keikavoussi, P., Agaimy, A., Kiesewetter, F., Hartmann, A., and Schneider-Stock, R. (2013) Mutation landscape in melanoma patients clinical implications of heterogeneity of BRAF mutations, British Journal of Cancer 109, 2833-2841.
[4] Martinez-Garza, S. G., Nunez-Salazar, A., Calderon-Garciduenas, A. L., Bosques-Padilla, F. J., Niderhauser-Garcia, A., and Barrera-Saldana, H. A. (1999) Frequency and clinicopathology associations of K-ras mutations in colorectal cancer in a northeast Mexican population, Digestive Diseases 17, 225-229.
[5] Rasuck, C. G., Leite, S. M. O., Komatsuzaki, F., Ferreira, A. C. S., Oliveira, V. C., and Gomes, K. B. (2012) Association between methylation in mismatch repair genes, V600E BRAF mutation and microsatellite instability in colorectal cancer patients, Mol. Biol. Reports 39, 2553-2560.
[6] Dahan, L., Huang, L., Kedmi, R., Behlke, M. A., and Peer, D. (2013) SNP detection in mRNA in living cells using allele specific FRET probes, PloS one 8, e72389.
[7] Bos, J. L. (1989) Ras oncogenesis in human cancer—a review Cancer Res. 49, 4682-4689.
[8] Pellegata, N. S., Sessa, F., Renault, B., Bonato, M., Leone, B. E., Solcia, E., and Ranzani, G. N. (1994) K-ras and P53 gene mutations in pancreatic cancer—ductal and nonductal tumors progress through different genetic lesions Cancer Res. 54, 1556-1560.
[9] Yilmaz, A., Mohamed, N., Patterson, K. A., Tang, Y., Shilo, K., Villalona-Calero, M. A., Davis, M. E., Zhou, X.-P., Frankel, W., Otterson, G. A., and Zhao, W. (2014) Clinical and Metabolic Parameters in Non-Small Cell Lung Carcinoma and Colorectal Cancer Patients with and without KRAS Mutations, Inl. J. Environmen. Res. Public Health 11, 8645-8660.
[10] Santangelo, P. J., Nix, B., Tsourkas, A., and Bao, G. (2004) Dual FRET molecular beacons for mRNA detection in living cells, Nucleic Acids Res. 32.
[11] Wang, Z., Zhang, K., Shen, Y., Smith, J., Bloch, S., Achilefu, S., Wooley, K. L., and Taylor, J.-S. (2013) Imaging mRNA expression levels in living cells with PNA center dot DNA binary FRET probes delivered by cationic shell-crosslinked nanoparticles, Org. Biomol. Chem. 11, 3159-3167.
[12] Abe, H., and Kool, E. T. (2006) Flow cytometric detection of specific RNAs in native human cells with quenched autoligating FRET probes, Proc. Natl. Acad. Sci. U.S.A 103, 263-268.
[13] Okabe, K., Harada, Y., Zhang, J., Tadakuma, H., Tani, T., and Funatsu, T. (2011) Real time monitoring of endogenous cytoplasmic mRNA using linear antisense 2′-O-methyl RNA probes in living cells, Nucleic Acids Res. 39, e20/21-e20/13.
[14] Bratu, D. P., Cha, B. J., Mhlanga, M. M., Kramer, F. R., and Tyagi, S. (2003) Visualizing the distribution and transport of mRNAs in living cells, Proc. Natl. Acad. Sci. U.S.A 100, 13308-13313.
[15] Catrina, I. E., Marras, S. A. E., and Bratu, D. P. (2012) Tiny Molecular Beacons: LNA/2′-O-methyl RNA Chimeric Probes for Imaging Dynamic mRNA Processes in Living Cells, ACS Chem. Biol. 7, 1586-1595.
[16] Chen, T., Wu, C. S., Jimenez, E., Zhu, Z., Dajac, J. G., You, M., Han, D., Zhang, X., and Tan, W. (2013) DNA Micelle Flares for Intracellular mRNA Imaging and Gene Therapy, Angew. Chem. Int. Ed. 52, 2012-2016.
[17] Nitin, N., and Bao, G. (2008) NLS Peptide Conjugated Molecular Beacons for Visualizing Nuclear RNA in Living Cells, Bioconjugate Chem. 19, 2205-2211.
[18] Peng, X. H., Cao, Z. H., Xia, J. T., Carlson, G. W., Lewis, M. M., Wood, W. C., and Yang, L. (2005) Real-time detection of gene expression in cancer cells using molecular beacon imaging: New strategies for cancer research, Cancer Res. 65, 1909-1917.
[19] Qiu, L., Wu, C., You, M., Han, D., Chen, T., Zhu, G., Jiang, J., Yu, R., and Tan, W. (2013) A Targeted, Self-Delivered, and Photocontrolled Molecular Beacon for mRNA Detection in Living Cells, J. Am. Chem. Soc. 135, 12952-12955.
[20] Dong, H., Ding, L., Yan, F., Ji, H., and Ju, H. (2011) The use of polyethylenimine-grafted graphene nanoribbon for cellular delivery of locked nucleic acid modified molecular beacon for recognition of microRNA, Biomaterials 32, 3875-3882.
[21] Chen, A. K., Davydenko, O., Behlke, M. A., and Tsourkas, A. (2010) Ratiometric bimolecular beacons for the sensitive detection of RNA in single living cells, Nucleic Acids Res. 38, e148.
[22] Zhang, X., Song, Y., Shah, A. Y., Lekova, V., Raj, A., Huang, L., Behlke, M. A., and Tsourkas, A. (2013) Quantitative assessment of ratiometric bimolecular beacons as a tool for imaging single engineered RNA transcripts and measuring gene expression in living cells, Nucleic Acids Res. 41, 1-12.
[23] Karlsen, K. K., Okholm, A., Kjems, J., and Wengel, J. (2013) A quencher-free molecular beacon design based on pyrene excimer fluorescence using pyrene-labeled UNA (unlocked nucleic acid), Bioorg. Med Chem. 21, 6186-6190.
[24] Waki, R., Yamayoshi, A., Kobori, A., and Murakami, A. (2011) Development of a system to sensitively and specifically visualize c-fos mRNA in living cells using bispyrene-modified RNA probes, Chem. Comm. 47, 4204-4206.
[25] Asanuma, H., Akahane, M., Niwa, R., Kashida, H., and Kamiya, Y. (2015) Highly sensitive and robust linear probe for detection of mRNA in cells, Angew. Chem., Int. Ed. 54, 4315-4319.
[26] Cheglakov, Z., Cronin, T. M., He, C., and Weizmann, Y. (2015) Live Cell MicroRNA Imaging Using Cascade Hybridization Reaction, J. Am. Chem. Soc. 137, 6116-6119.
[27] Wu, Z., Liu, G.-Q., Yang, X.-L., and Jiang, J.-H. (2015) Electrostatic nucleic acid nanoassembly enables hybridization chain reaction in living cells for ultrasensitive mRNA imaging, J. Am. Chem. Soc. 137, 6829-6836.
[28] Halo, T. L., McMahon, K. M., Angeloni, N. L., Xu, Y., Wang, W., Chinen, A. B., Malin, D., Strekalova, E., Cryns, V. L., Cheng, C., Mirkin, C. A., and Thaxton, C. S. (2014) NanoFlares for the detection, isolation, and culture of live tumor cells from human blood, Proc. Natl. Acad. Sci. U.S.A 111, 17104-17109.
[29] Prigodich, A. E., Randeria, P. S., Briley, W. E., Kim, N. J., Daniel, W. L., Giljohann, D. A., and Mirkin, C. A. (2012) Multiplexed Nanoflares: mRNA Detection in Live Cells, Anal. Chem. 84, 2062-2066.
[30] Prigodich, A. E., Seferos, D. S., Massich, M. D., Giljohann, D. A., Lane, B. C., and Mirkin, C. A. (2009) Nano-flares for mRNA regulation and detection, ACS Nano 3, 2147-2152.
[31] Jayagopal, A., Halfpenny, K. C., Perez, J. W., and Wright, D. W. (2010) Hairpin DNA-Functionalized Gold Colloids for the Imaging of mRNA in Live Cells, J. Am. Chem. Soc. 132, 9789-9796.
[32] Li, N., Chang, C., Pan, W., and Tang, B. (2012) A Multicolor Nanoprobe for Detection and Imaging of Tumor-Related mRNAs in Living Cells, Angew. Chem. Int. Ed. 51, 7426-7430.
[33] Pan, W., Zhang, T., Yang, H., Diao, W., Li, N., and Tang, B. (2013) Multiplexed Detection and Imaging of Intracellular mRNAs Using a Four-Color Nanoprobe, Anal. Chem. 85, 10581-10588.
[34] Qiao, G., Gao, Y., Li, N., Yu, Z., Zhuo, L., and Tang, B. (2011) Simultaneous Detection of Intracellular Tumor mRNA with Bi-Color Imaging Based on a Gold Nanoparticle/Molecular Beacon, Chem.—Eur. J. 17, 11210-11215.
[35] Harry, S. R., Hicks, D. J., Amiri, K. I., and Wright, D. W. (2010) Hairpin DNA coated gold nanoparticles as intracellular mRNA probes for the detection of tyrosinase gene expression in melanoma cells, Chem. Comm. 46, 5557-5559.
[36] Bethge, L., Jarikote, D. V., and Seitz, O. (2008) New cyanine dyes as base surrogates in PNA: Forced intercalation probes (FIT-probes) for homogeneous SNP detection, Bioorg. Med. Chem. 16, 114-125.
[37] Koehler, O., Jarikote, D. V., and Seitz, O. (2005) Forced intercalation probes (FIT probes): Thiazole orange as a fluorescent base in peptide nucleic acids for homogeneous single-nucleotide-polymorphism detection, Chem Bio Chem 6, 69-77.
[38] Kummer, S., Knoll, A., Socher, E., Bethge, L., Herrmann, A., and Seitz, 0. (2011) Fluorescence Imaging of Influenza H1N1 mRNA in Living Infected Cells Using Single-Chromophore FIT-PNA, Angew. Chem. Int. Ed. 50, 1931-1934.
[39] Kummer, S., Knoll, A., Sucher, E., Bethge, L., Herrmann, A., and Seitz, O. (2012) PNA FIT-Probes for the Dual Color Imaging of Two Viral mRNA Targets in Influenza H1N1 Infected Live Cells, Bioconjugate Chem. 23, 2051-2060.
[40] Socher, E., Bethge, L., Knoll, A., Jungnick, N., Herrmann, A., and Seitz, O. (2008) Low-noise stemless PNA beacons for sensitive DNA and RNA detection, Angew. Chem., Int. Ed. 47, 9555-9559.
[41] Socher, E., Jarikote, D. V., Knoll, A., Roeglin, L., Burmeister, J., and Seitz, O. (2008) FIT probes: Peptide nucleic acid probes with a fluorescent base surrogate enable real-time DNA quantification and single nucleotide polymorphism discovery, Anal. Biochem. 375, 318-330.
[42] Silva, G. L., Ediz, V., Yaron, D., and Armitage, B. A. (2007) Experimental and computational investigation of unsymmetrical cyanine dyes: Understanding torsionally responsive fluorogenic dyes, J. Am. Chem. Soc. 129, 5710-5718.
[43] Kam, Y., Rubinstein, A., Nissan, A., Halle, D., and Yavin, E. (2012) Detection of endogenous K-ras mRNA in living cells at a single base resolution by a PNA molecular beacon, Molecular Pharmaceutics 9, 685-693.
[44] Sonar, M. V., Wampole, M. E., Jin, Y.-Y., Chen, C.-P., Thakur, M. L., and Wickstrom, E. (2014) Fluorescence Detection of KRAS2 mRNA Hybridization in Lung Cancer Cells with PNA-Peptides Containing an Internal Thiazole Orange, Bioconjugate Chem. 25, 1697-1708.
[45] Hovelmann, F., Gaspar, I. Chamiolo, J., Kasper, M., Steffen, J., Ephrussi, A., and Seitz, 0. (2015) LNA-enhanced DNA FIT-probes for multicolour RNA imaging, Chem. Sci. 7, 128-135.

### BACKGROUND

In the past decades much effort has been put forth in the discovery of biomarkers in cancer as means for designing novel drug targets and for diagnostic purposes¹.

Using RNA as a biomarker has the advantage that detection could, in theory, be based not only on the over-expression of a given RNA molecule in malignant cells but also on the discrimination between mutated and non-mutated transcripts that are manifested in many types of malignancies²⁻⁶. In this regard, the KRAS oncogene is an important biomarker since it is activated in many types of adenocarcinomas⁷ and has frequent single base mutations associated with early stages of tumorogenesis⁸. For example, 90% of the activating mutations in colorectal cancer are found in codon 12 (wild-type-GGT)⁴ and the most frequently observed types of mutations are G to A transitions⁹. Therefore, KRAS was selected as a model gene for detection of mRNA in malignant cell lines.

Most of the current RNA detection systems are based on sequence specific hybridization of a labeled oligonucleotide (ODN)-probe and a target genetic marker. A wide range of labeling molecules for real time detection have been studied; many, based on fluorescent probes. Several classes of fluorescent ODNs have been developed for RNA detection in living cells, such as fluorescence resonance energy transfer (FRET) based oligonucleotides¹⁰⁻¹³, dual-labelled hairpin oligonucleotides (e.g. molecular beacons¹⁴⁻²⁰ and ratiometric biomolecular beacons (RBMB)^{21, 22}), Pyrene-modified oligonucleotides^{23, 24}, Perylene-modified oligonucleotides²⁵, Hybridization Chain Reaction (HCR) probes^{26, 27} and Au NPs modified oligonucleotides²⁸⁻³⁵.

PNA molecules containing the cyanine dye thiazole orange (TO) as well as other cyanines were used as replacement of a canonical nucleobase³⁶⁻⁴¹. These forced intercalation probes (FIT-probes) exhibit a remarkable fluorescence enhancement upon hybridization to their target DNA/RNA sequence, making them suitable for sequence specific detection of RNA and DNA.

These PNA oligomers are based on the property of mono-methine cyanine dyes, containing a flexible methine bond, which contributes to a non-planar conformation and non-radiative decay of the dye molecule⁴². Upon intercalation within dsDNA, the TO adopts a planar conformation and therefore becomes strongly fluorescent. Additionally, it was shown, that the enhancement of fluorescence by TO-based FIT probes is highly sensitive to localized perturbations of the duplex structure such as those imposed by an adjacent base mismatch, allowing the detection of SNP³⁷. It was previously published that it is possible to detect cancer using biomarker by targeting mutant KRAS^{43, 44} and that this mRNA transcript can be detected and discriminated at a single nucleotide resolution in living cells.

Nonetheless, TO and other related cyanine dyes absorb light in the visible region from ca. 500 nm (TO) to lower wavelengths³⁶. Hovelmann F. et al. showed a BisQ probe that was introduced to a DNA-LNA oligo and used to detect mRNA in developing oocytes from Drosphila melanogaster⁴⁵. These spectral properties are not suitable for in-vivo or in-situ imaging because tissue (e.g. hemoglobin) and cells auto-fluorescence at these wavelengths.

Thus, there is still a need for fast, straightforward and sensitive single mismatch or match based diagnosis and imaging of disease and disorders in living cells and tissue.

### General Description

The inventors of the present application have found new conjugates for use as a sensitive probe for diagnosing, imaging, and treating diseases and disorders.

The present invention provides a conjugate comprising (a) at least one red to NIR (600-790 nm) emitting probe component connected to (b) at least one complementary component.

The term “complementary component” should be understood to encompass any moiety that is complementary with a target nucleic acid sequence in a cell or tissue (said cell or tissue could be the cell or tissue of a subject, a cell or tissue of a parasite, a cell or tissue of an organism and so forth). In some embodiments, said at least one complementary component is selected from a Peptide nucleic acid (PNA), a DNA sequence and an RNA sequence and any combinations thereof. In some further embodiments said complementary component is a peptide nucleic acid (PNA). In other embodiments said complementary component is DNA sequence. In further embodiments, said complementary component is an RNA sequence. In some further embodiments, said target sequence is indicative of a mutation, a condition or disease. In other embodiments, said target sequence is indicative of the presence of an organism (such as for example a parasite) in a host subject being administered with said conjugate. In other embodiments, said target sequence is indicative of an acquired genetic resistance of an organism to a substance (such as for example a drug).

The term “Peptide Nucleic Acid (PNA)” should be understood to encompass a nucleotide sequence comprising between 10 to 25 base nucleotide (in some embodiments between 16 to 18 bases), designed to be complementary with a specific sequence (such as a sequence in a gene or an oncogene) or a mutation thereof, that is indicative of a condition, an acquired resistance to a substance, a disorder or a disease (all of which are genetically manifested).

In some embodiments, the complementary component (such as for example PNA) is designed to be complementary to a mutation of an oncogene. Such mutations of oncogenes are known to be associated with specific malignant processes and diseases. For example, a specific mutation of the KRAS oncogene is associated with colon cancer. Thus, a complementary component (e.g. PNA) designed to be complementary to a specific mutation of the KRAS oncogene, can be used with a conjugate of the invention for the purpose of diagnosing and imaging of said colon cancer cell.

The term “red to NIR (near infra red) emitting probe component” refers to a moiety of a compound that possesses fluorescent spectral properties within the red to NIR (in some embodiments far red) radiation spectrum in the wavelength range of between about 600 nm to about 790 nm upon any change in the physical or chemical properties of the moiety, including but not limited to: change in the structural conformation of the moeity, change in the connectivity of the moeity to the complementary component, change in the steric degrees of freedom of the component. Such changes in the physical and/or chemical properties of the probe moeity come about due to the hybridization of the complementary component of the conjugate of the invention with the target sequence of interest in a cell (such as for example known DNA/RNA sequences indicative of a condition or a disease). In some embodiments said probe is a red to far red emitting probe component, having a radiation spectrum in the wavelength range of between about 600 nm to about 750 nm.

The term “hybridisation” refers to the bonding interaction between the complementary component of the conjugate and a target sequence in the cell. Upon hybridization of complementary component of the conjugate with the target sequence (such as a DNA or RNA sequences) there is a significant fluorescence enhancement at the red to NIR range, relative to complementary component emission in non hybridised (single stranded) form.

In some embodiments, said red to NIR emitting probe component is emitting radiation in the wavelength range of 600 nm to 790 nm. In some embodiments, said probe component is emitting radiation in the range of 600 nm to 750 nm. In some embodiments, said probe component is emitting radiation in the range of 610 nm to 700 nm. In some embodiments, said probe component is emitting radiation in the range of 610 nm to 770 nm. In some embodiments, said probe component is emitting radiation in the range of 610 nm to 790 nm. In some embodiments, said probe component is emitting radiation in the range of 680 nm to 790 nm. In some embodiments, said probe component is emitting radiation in the range of 575 nm-790 nm. In some embodiments, the wavelength of said probe component of 575 nm-790 nm is measured prior to the conjugation of said probe into the conjugate (i.e. as a stand alone molecule wherein the complementary probe is substituted with for example H). In some embodiments, the wavelength of said probe component of 575 nm-600 nm is measured prior to the conjugation of said probe into the conjugate (i.e. as a stand alone molecule wherein the complementary probe is substituted with for example H).

In some embodiments red to far-red emitting probe component comprises at least one bond that changes its confirmation due to hybridization of the complementary component of the conjugate of the invention, with the sequence of interest at the target cell. In some embodiments, red to NIR emitting probe component comprises one methine bond.

In some embodiments a red to NIR emitting probe component is selected from the following compounds:

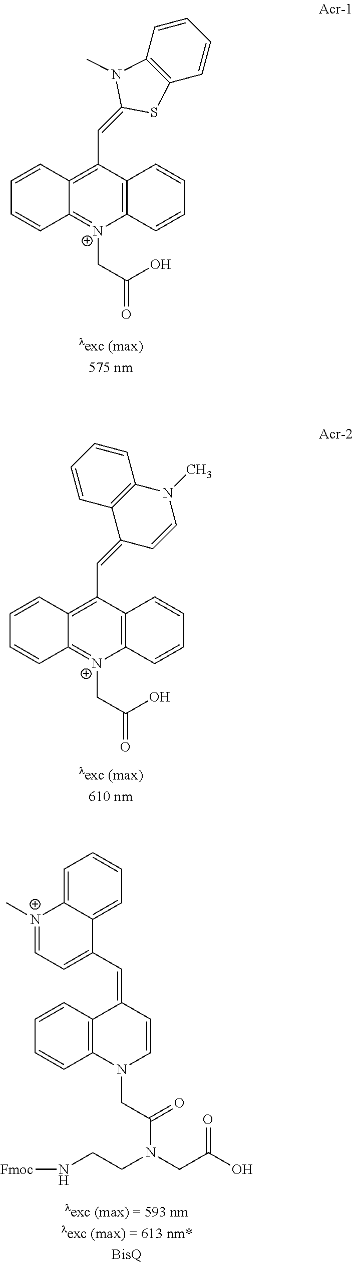

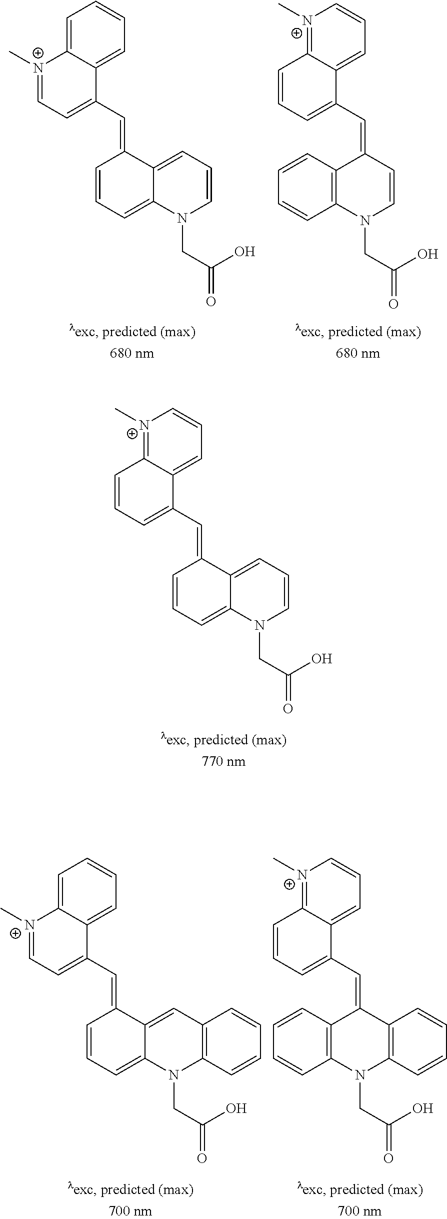 [SMILES: C[n+]1ccc(/C=c2\cccc3c2=CC=CN3CC(=O)O)c2ccccc21 ; C[n+]1cccc2c(/C=C3/C=CN(CC(=O)O)c4ccccc43)cccc21 ; C[n+]1cccc2c(/C=c3\cccc4c3=CC=CN4CC(=O)O)cccc21 ; C[n+]1ccc(/C=c2\cccc3c2=Cc2ccccc2N3CC(=O)O)c2ccccc21 ; C[n+]1cccc2c(C=C3c4ccccc4N(CC(=O)O)c4ccccc43)cccc21]

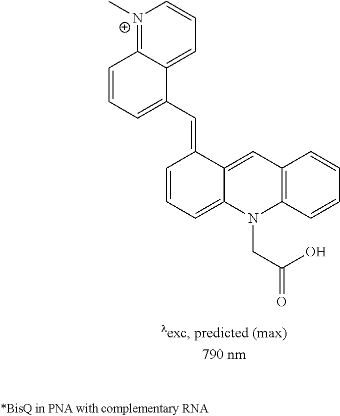 [SMILES: C[n+]1cccc2c(/C=c3\cccc4c3=Cc3ccccc3N4CC(=O)O)cccc21]

The term “conjugate” refers to a compound comprising at least the two components described above (i.e. red to NIR emitting probe component moiety and complementary component) connected to each other at any position of each component, through any type of bond including chemical bond, coordination bond, hydrogen bond and so forth.

For example, scheme 1 below provides a procedure for the preparation of a conjugate of the invention wherein a long wavelength emitting probe (LWEP) molecule (680 nm) is reacted so as to connect to a PNA sequence targeting the kRAS oncogene.

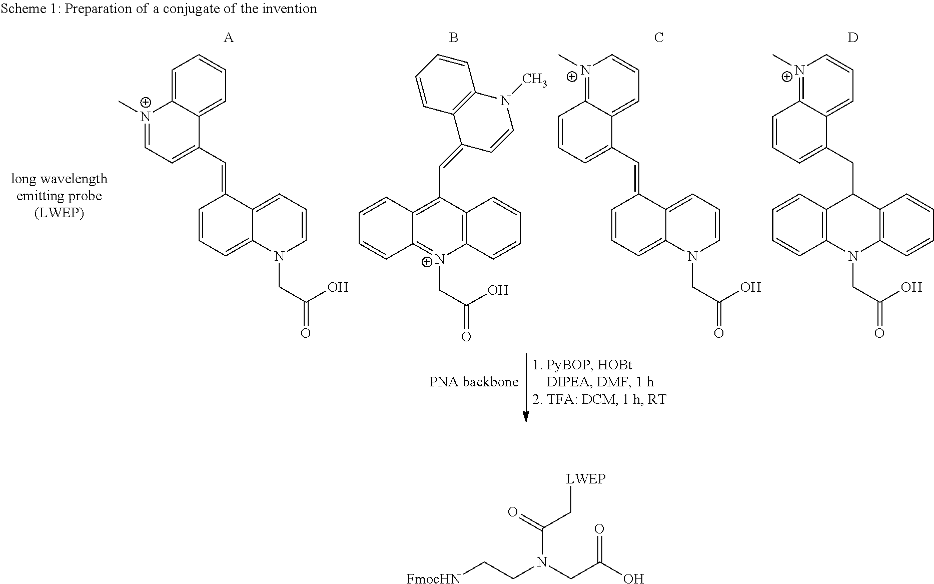

In some embodiments a conjugate of the invention is designed in a way that the complementary component is an oligonucleotide sequence complementary to a targeted sequence.

The conjugate of the invention exhibits fluorescence enhancement at a red to NIR spectrum upon hybridization of the complementary component of the conjugate to a targeted sequence in living cells, tissue or organism, it is designed to complement. The enhancement fluorescence at a red to NIR spectrum is due to the conformational changes of the red to NIR emitting probe component of the conjugate of the invention.

It is important to note that such changes that provide the fluorescence enhancement at a red to NIR spectrum can occur only upon the complete match between the complementary component and the target sequence in the cell, tissue or organism. Due to the exact complementarity of the complementary component and the exact design of the sequence thereof the conjugate of the invention is capable of detecting specific sequences in living cells or other cells, at single nucleotide polymorphism (SNP) resolution.

In some embodiments a complementary component comprises a sequence complementary to the targeted nucleic acid sequence. In some embodiments, said target nucleic acid sequence is present in a living cell. In other embodiments, the cell is present in a living organism. The target nucleic acid sequence may be a genomic sequence (coding, regulatory or non coding DNA sequence, or an RNA (mRNA, iRNA, microRNA)). In some embodiments, said genomic sequence is a DNA sequence. In some other embodiments, said sequence is an RNA sequence.

Non limiting examples of target sequences are as follows: KRAS, kRAS, abl, Af4/hrx, akt-2, alk, alk/npm, aml1, aml1/mtg8, axl, bc1-2, 3, 6, bcr/abl, c-myc, dbl, dek/c an, E2A/pbx1, egfr, enl/hrx, erg/TLS, erbB, erbB-2, ets-1, ews/fli-1, fms, fos, fps, gli, gsp, HER2/neu, hox11, hst, IL-3, int-2, jun, kit, KS3, K-sam, Lbc, lck, lmo1, lmo2, L-myc, lyl-1, lyt-10, lyt-10/C alpha1, mas, mdm-2, mll, mos, mtg8/aml1, myb, MYH11/CBFB, neu, N-myc, ost, pax-5, pbx1/E2A, pim-1, PRAD-1, raf, RAR/PML, rash, rasN, rel/nrg, ret, rhom1, rhom2, ros, ski, sis, set/can, src, tal1, tal2, tan-1, Tiam1, TSC2, trk.

In some embodiments, the complementary component is designed to contain a sequence with different mutations that are complementary to known mutations in genes or oncogenes. In some examples provided below the oncogene is KRAS oncogene and known mutation thereof are indicative of pancreas cancer.

In some embodiments, the complementary component is designed to be complementary with a gene sequence that is associated with a disorder, an acquired resistance to a particular substance, a condition or a disease. In some embodiments, said gene sequence is a mutated gene sequence.

In some embodiments a BisQ-PNA conjugate is designed in a way that the PNA sequence is complementary to the targeted gene sequence of KRAS or kRAS. In further embodiments a BisQ-PNA conjugate is designed in a way that the PNA sequence is complementary to the targeted gene sequence of other oncogenes.

In some embodiments a conjugate of the invention further comprises at least one moiety designed for cellular internalization. In some embodiments, said at least one moiety designed for cellular internalization is an amino acid sequence. In some embodiments said additional sequence comprises four D-lysines (for example at the PNA's C-terminus). In other embodiments, said at least one moiety designed for cellular internalization a fatty acid derivative (such as for example stearyl fatty acid).

The term “cellular internalization” refers to the ability of the conjugate of the invention to enter the cell barrier. Another term for internalization is endocytosis, in which a conjugate of the invention is capable of being engulfed by the cell membrane and drawn into the cell. This is aided and made more efficient by the use of the above defined moieties.

Also included within the scope of the invention is a conjugate, further comprising at least one chemotherapeutic agent, thus providing targeted treatment of cancer directly at the cellular level.

In some embodiment a conjugate further comprises at least one isotopically labeled antibody, thus providing targeted radioimmunotherapy wherein a radiative energy is directly given into the targeted cancer cells, using a monoclonal antibody carrier.

The invention further provides a composition comprising at least one conjugate as defined herein above and below.

In some further aspect the invention provides a conjugate as defined herein above and below for use in diagnosis and imaging of at least one malignant condition or disease.

The term “malignant condition or disease” refers to any cancerous condition or disease that is presented in abnormal cell growth capable of invading into adjacent tissues, and may be capable of spreading to distant tissues. Such conditions and disease include, but are not limited to: Adrenocortical carcinoma, Bladder cancer, Bone cancer, Osteosarcoma, Malignant fibrous histiocytoma, Breast cancer, Burkitt lymphoma, Carcinoid tumour, Cerebellar astrocytoma, Cerebral astrocytoma/Malignant glioma, childhood, Cervical cancer, Colon Cancer, Cutaneous T-cell lymphoma, Desmoplastic small round cell tumour, Endometrial cancer, Ependymoma, Oesophageal cancer, Ewing's sarcoma, Extragonadal Germ cell tumour, Extrahepatic bile duct cancer, Eye Cancer, Retinoblastoma, Gallbladder cancer, Head and neck cancer, Heart cancer, Hepatocellular cancer, Hodgkin lymphoma, Hypopharyngeal cancer, Intraocular Melanoma, Islet Cell Carcinoma, Kaposi sarcoma, Laryngeal Cancer, Liver Cancer, Lung Cancer, Lymphomas, Medulloblastoma, Melanoma, Merkel Cell Carcinoma, Mesothelioma, Mouth Cancer, Mycosis Fungoides, Nasopharyngeal carcinoma, Neuroblastoma, Non-small cell lung cancer, Oropharyngeal cancer, Ovarian cancer Pancreatic cancer, Parathyroid cancer, Penile cancer, Pharyngeal cancer, Pleuropulmonary blastoma, Prostate cancer, Rectal cancer, Renal cell carcinoma, Retinoblastoma, Rhabdomyosarcoma, Salivary gland cancer, Stomach cancer, Testicular cancer, Throat cancer, Thymic carcinoma, Thyroid cancer, Urethral cancer, Uterine cancer, endometrial, Uterine sarcoma, Wilms tumour.

The term “diagnosis” refers to any type of medical diagnosis of determining the existence and state of a disease or condition in a subject, whether the subject has shown symptoms of any condition or disease or not (in case of a routine diagnosis procedure due to risk factors or age). Said diagnosis can be performed in vivo (administered a conjugate or composition of the invention to a subject and subjecting said subject to diagnosing devices and methods), or said diagnosis can be performed ex vivo (on a bodily sample taken from said subject, either priro or after being administered with a conjugate or composition of the invention).

The term “imaging” refers to the creation of visual representations of the interior of a body for clinical analysis and medical intervention. Using this technique with the conjugate of the invention will allow for the determination of the location and extent of a disease or condition (for example malignant condition) in a subject administered with said conjugate and exposed to red-to NIR fluorescence radiation.

The invention further provides a conjugate of the invention for use in the diagnosis of at least one genetic condition, disorder or disease.

The term “genetic condition, disorder or disease” should be understood to encompass any condition, disorder or disease that is caused by one or more abnormalities in the genome of a subject or organism. In some embodiments, said genetic condition, disorder to disease are present from birth (congenital). In some embodiments, said genetic disorder, condition or disease is hereditary. In other embodiments, said genetic condition, disorder or disease is caused by new mutations or changes to the DNA of the organism or subject. In some embodiments, said genetic condition, disorder or disease is a single gene mutation (autosomal dominant, autosomal recessive, X-linked dominant, X-linked recessive, Y-linked, mitochondrial). In other embodiments, said genetic condition, disorder or disease are polygenic. Multifactorial disorders, conditions and disease include, but are not limited to: heart disease, diabetes, asthma, autoimmune diseases such as multiple sclerosis, cancers, ciliopathies, cleft palate, hypertension, inflammatory bowel disease, intellectual disability, mood disorder, obesity, refractive error, infertility and so forth. None limiting examples of such diseases and disorders include: DiGeorge syndrome, Angelman syndrome, Canavan disease, Charcot-Marie-Tooth disease, Cri du chat, Cystic fibrosis, Down Syndrome, Duchenne muscular dystrophy, Haemophilia, Klinefelter syndrome, Neurofibromatosis, Phenylketonuria, Prader-Willi syndrome, Sickle-cell disease, Tay-Sachs disease, Turner syndrome. In some embodiments, said genetic condition, disorder or disease can be a condition associated with single nucleotide polymorphism (SNP).

In some further aspect, the invention provides a conjugate as discloses herein above and below for use in prenatal diagnosis of a genetic condition or disease.

The term “prenatal diagnosis” refers to of diagnosis of a condition or disease in a fetus before delivery, wherein said diagnosis is performed on cells derived from the placental villae or the amniotic fluid of said pregnant mother. Such diagnosis using a conjugate of the invention may be performed as early as 10-12 weeks' from gestation providing sensitive and accurate results in a short period of time. In other embodiments, said prenatal diagnosis is performed at 15-18 weeks' from gestation providing sensitive and accurate results in a short period of time.

The invention further provides a method for detecting a genetic condition or disease in a fetus comprising the steps of incubating a sample of fetal living cells or living tissue with a conjugate of the invention (wherein said PNA component is designed to have a particular known sequence which is complementary to the mutated sequence of interest or a sequence that is indicative of a genetic condition); exposing the incubated cells to a red to far red detector and a fluorescent signal is detected at the red to far red spectrum. In case the sample cells or tissue derived from the fetus contain the target sequence associated with a genetic condition or disease, the red to NIR emitting probe component will emit light in the red to NIR region upon exposure to red-to NIR radiation due to the hybridization of the complementary component and target sequence. In case red to far red emission is detected, this provides diagnosis of a genetic condition or disease of said fetus. In case no hybridization of the complementary component was achieved upon incubation of the sample with a conjugate of the invention, then the fetus is diagnosed as not having the genetic condition investigated. Such diagnosis using a conjugate of the invention may be performed as early as 10-12 weeks' from gestation providing sensitive and accurate results in a short period of time. In other embodiments, said prenatal diagnosis is performed at 15-18 weeks' from gestation providing sensitive and accurate results in a short period of time.

The invention further provides a conjugate as defined herein above and below for use in a method of in vitro diagnosis and imaging.

Under this method a sample tissue is excised from a subject and incubated with a conjugate of the invention, thereafter exposing said incubated tissue to red to NIR fluorescence detector, thereby diagnosing malignancy in said tissue. In case the sample cells or tissue excised from the subject contain the target sequence associated with a condition or disease, the red to NIR emitting probe component will emit light in the red to far red region upon exposure to red-to NIR radiation due to the hybridization of the complementary component and target sequence. In case red to NIR emission is detected, this provides diagnosis of a genetic condition or disease of said subject. In case no hybridization of the complementary component was achieved upon incubation of the sample with a conjugate of the invention, then the subject is diagnosed as not having the genetic condition investigated.

This invention further provides a conjugate as disclosed herein above and below for use in a method of in vivo diagnosis and imaging, i.e. diagnosis and imaging within the living body of a subject in a certain body part or tissue.

The invention provides a method of in vivo diagnosis and imaging of a condition or disorder comprising the steps of administering to a subject a conjugate of the invention, imaging at least a part of said subject's body using red-to far red fluorescence detector, thereby diagnosing malignant disorder in said subject.

In case the cells of the subject contain the target sequence associated with a condition or disease, the red to far red emitting probe component will emit light in the red to NIR region upon exposure to red-to NIR radiation due to the hybridization of the complementary component and target sequence. In case red to NIR emission is detected, this provides diagnosis of a condition or disease of said subject. In case no hybridization of the complementary component was achieved upon incubation of the sample with a conjugate of the invention, then the subject is diagnosed as not having the condition investigated.

In some embodiments of the invention the diagnosis and imaging of a condition or disorder in performed in living cells and tissue.

The term “living cells or living tissue” refers to any cells or tissue derived from or connected to a living organism comprising all cell and tissue components.

The invention also provides a conjugate as disclosed herein above and below for use in fluorescence guided surgery, i.e. is a diagnosis and imaging technique used to detect fluorescently labeled components during surgery. This technique allows for determining the extent of removal of malignant tissue during a malignancy removal surgery.

The invention provides a method for determining the extent of removal of malignant tissue during or after a malignancy removal surgery comprising the steps of removing a malignant tumor from a subject's body, incubating a conjugate of the invention (comprising a sequence complementary to a specific malignancy-associated mutation) with at least a part of the boarders of said removed malignant tissue (i.e. the outer perimeter of the excised tissue), exposing said incubated tissue to red-NIR fluorescence detector, thereby determining the extent of removal of malignant tissue. If the borders of said removed malignant tissue still emit fluorescent signal, this will indicate the presence of a remaining malignant tissue in said subject, which can be further removed.

The invention further includes a method for early diagnosis of a malignant disorder in a subject at risk comprising the steps of administering to a subject a conjugate of the invention, imaging at least a part of said subject's body using red-NIR fluorescence detector, thereby diagnosing malignant disorder of said subject.

The term “early diagnosis” refers to an early phase of establishing the existence, degree or metastasis condition of malignant disorder or disease of said individual, before a known symptom of malignancy appears. For example, if an individual undergoes colonoscopy and a polyp in his colon is detected and excised, testing for codon 12 mutations in the polyp tissue by the means of the present invention will diagnose whether the polyp is malignant or benign.

The invention further provides a kit comprising a conjugate as defined herein above and below, for use in the diagnosis of a genetic condition, disease or disorder, including instructions for use thereof.

In a further aspect the invention provides a kit comprising a conjugate as defined herein above and below, for use in the diagnosis of a malignant condition, disease or disorder, including instructions for use thereof.

In another one of its aspects the invention provides a kit comprising a conjugate as defined herein above and below, for use in the diagnosis of a mutated substance resistance of an organism, including instructions for use thereof. In another one of its aspects the invention provides a kit comprising a conjugate as defined herein above and below, for use in the diagnosis of a single nucleotide polymorphism of an organism, including instructions for use thereof.

The term “kit for use in diagnosis” or “diagnostic kit” should be understood to encompass an assembly of tools for use in diagnosing a condition, disease or disorder in a cell or tissue of a subject. The tools provided with the kit include, but are not limited to a composition comprising a conjugate of the invention and instructions for using said composition. Such instructions may include the sequence of operation of a device for detecting the conjugate of the invention in the cells or tissue of the subject, instructions on how to administer said composition to the subject and so forth. In some embodiments, said kit of the invention further comprises tools for administering a composition of the invention to a subject. In other embodiments, a kit of the invention comprises tools for sampling a body tissue from a subject for ex-vivo diagnosis of a condition, disease or disorder.

### BRIEF DESCRIPTION OF THE DRAWINGS

In order to better understand the subject matter that is disclosed herein and to exemplify how it may be carried out in practice, embodiments will now be described.

FIG. 1 depicts the ¹H NMR spectrum of BisQ in DMSO-d6.

FIG. 2. shows the HPLC chromatogram of PNA1. Eluents: A (0.1% TFA in water) and B (MeCN) were used in a linear gradient (11-40% B in 38 min) with a flow rate of 4 mL/min.

FIG. 3 shows the Maldi-TOF MS of PNA1. Mcaic=5148.26, Mobs=5148.26.

FIG. 4 shows the HPLC chromatogram of PNA2. Eluents: A (0.1% TFA in water) and B (MeCN) were used in a linear gradient (11-40% B in 38 min) with a flow rate of 4 mL/min.

FIG. 5 shows the Maldi-TOF MS of PNA2. Mcaic=5172.27, Mobs=5172.28.

FIG. 6. shows the UV-Vis spectra of TO and BisQ monomers.

FIG. 7. shows the UV-Vis spectrum of PNA1. Maximal absorption at 591 nm.

FIG. 8. shows the UV-Vis spectrum of PNA1:DNA. Maximal absorption at 587 nm.

FIG. 9. shows the UV-Vis spectrum of PNA2. Maximal absorption at 593 nm.

FIG. 10. shows the UV-Vis spectrum of PNA2:DNA. Maximal absorption at 593 nm.

FIGS. 11A-11B. show the fluorescence enhancement of PNA FIT probes after the addition of DNA. Fluorescence of PNA1 (FIG. 11A) and PNA2 (FIG. 11B) were recorded at 1.5 μM in buffered solution (black curve), with mmDNA (2 μM, dotted black curve), and with complementary DNA (2 μM, gray curve).

FIG. 12. shows the fluorescence microscopy images of Panc-1, HT-29, and Bxpc-3 cells incubated for 3 hours at 370 C with 0.5 μM PNA2. Lower panel shows the red emission solely in Panc-1 cells.

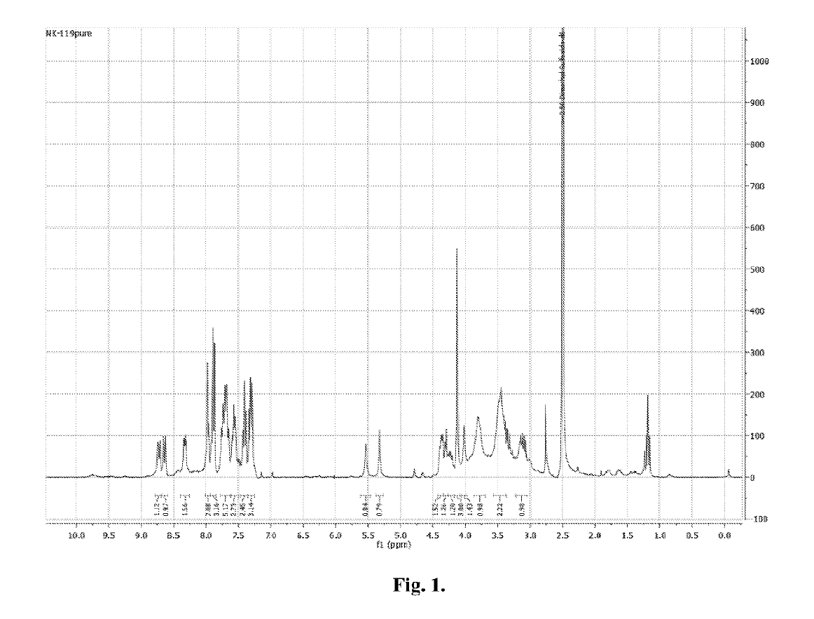

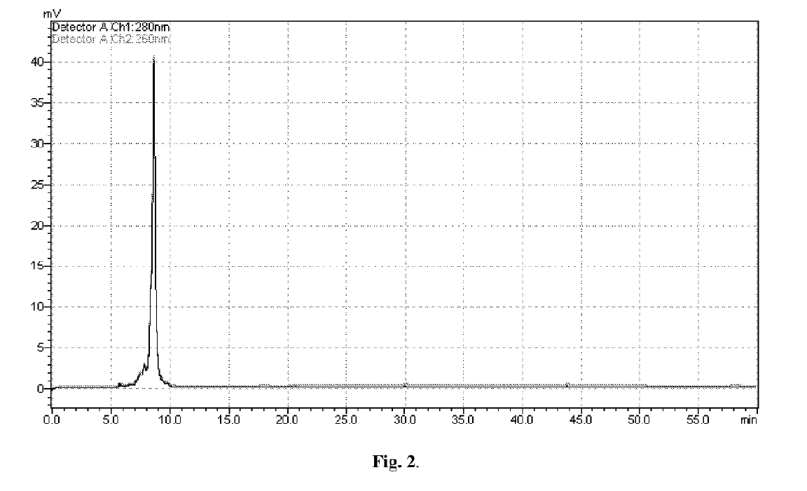

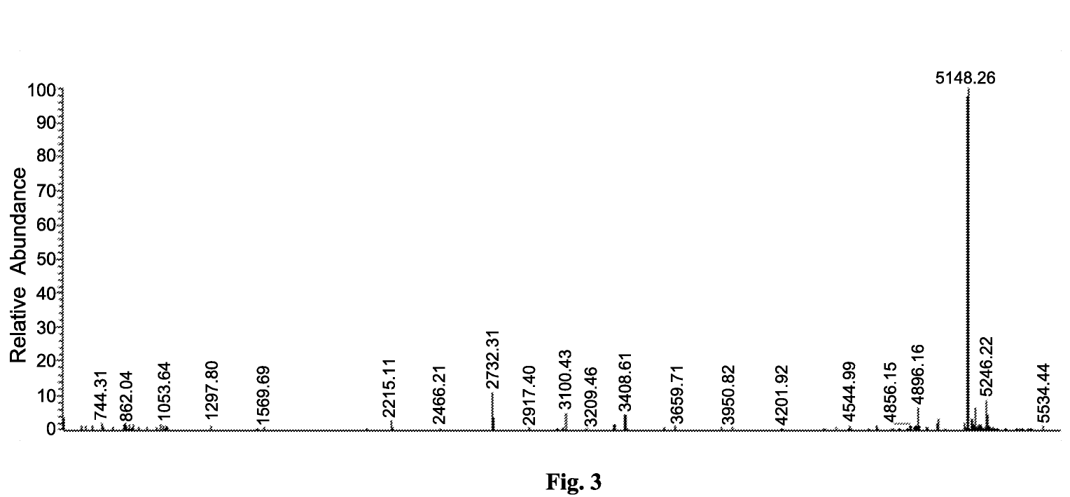

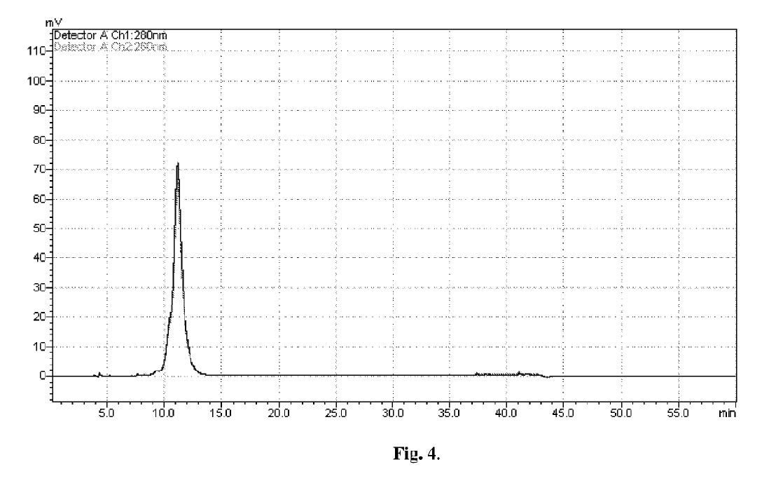

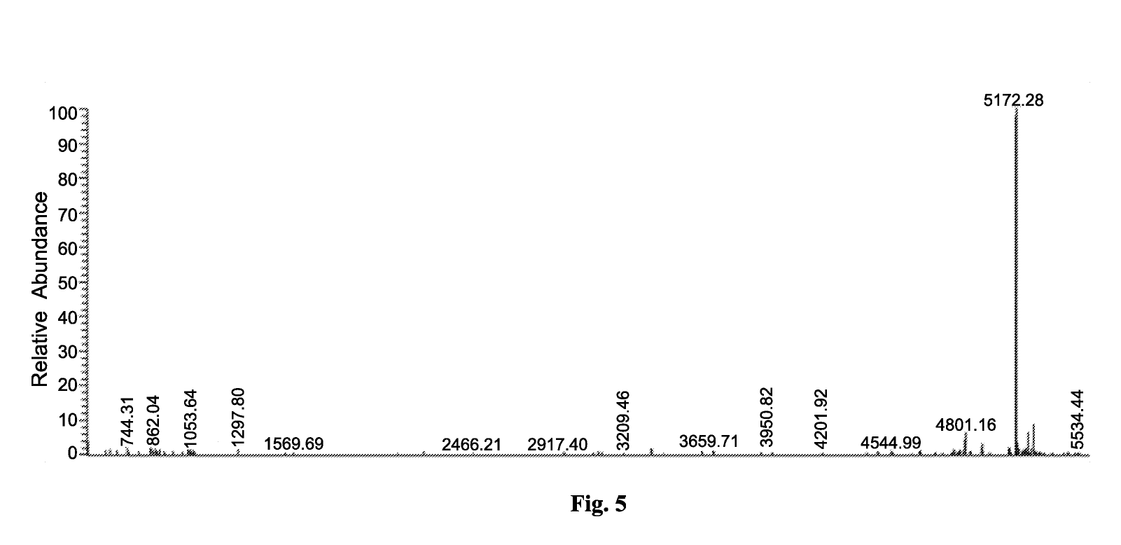

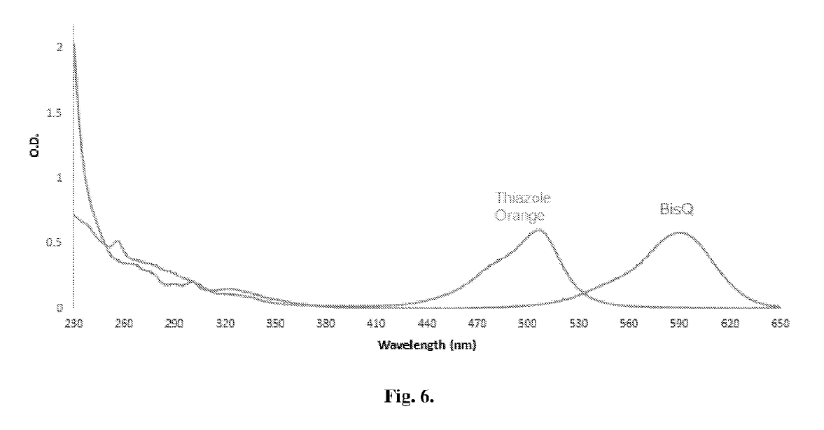

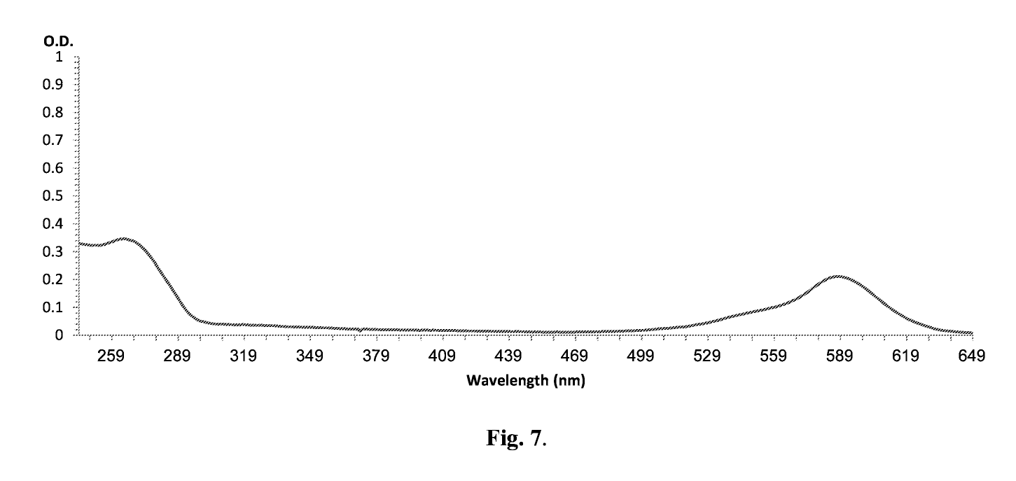

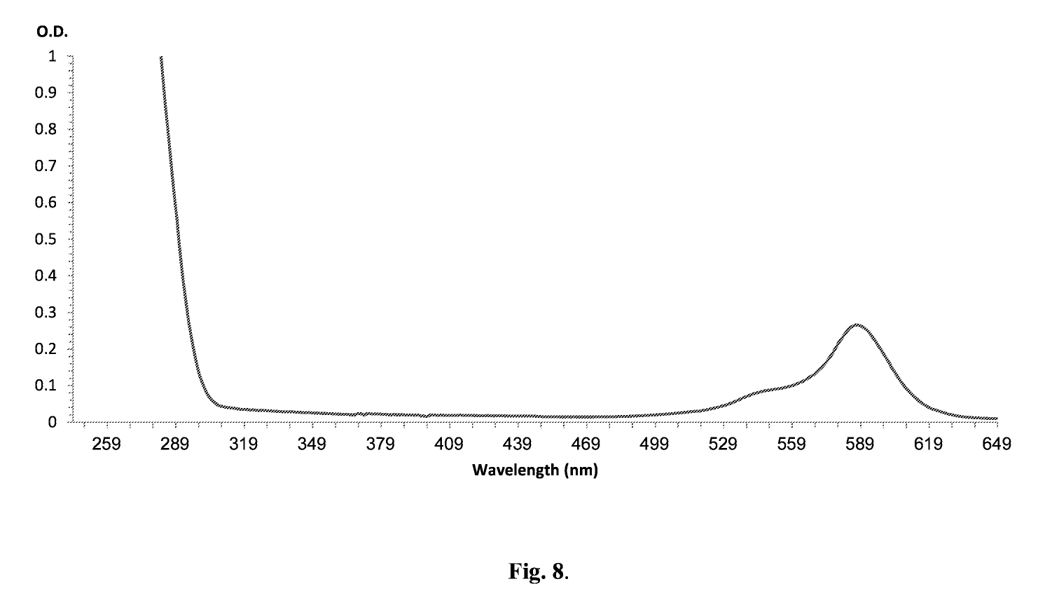

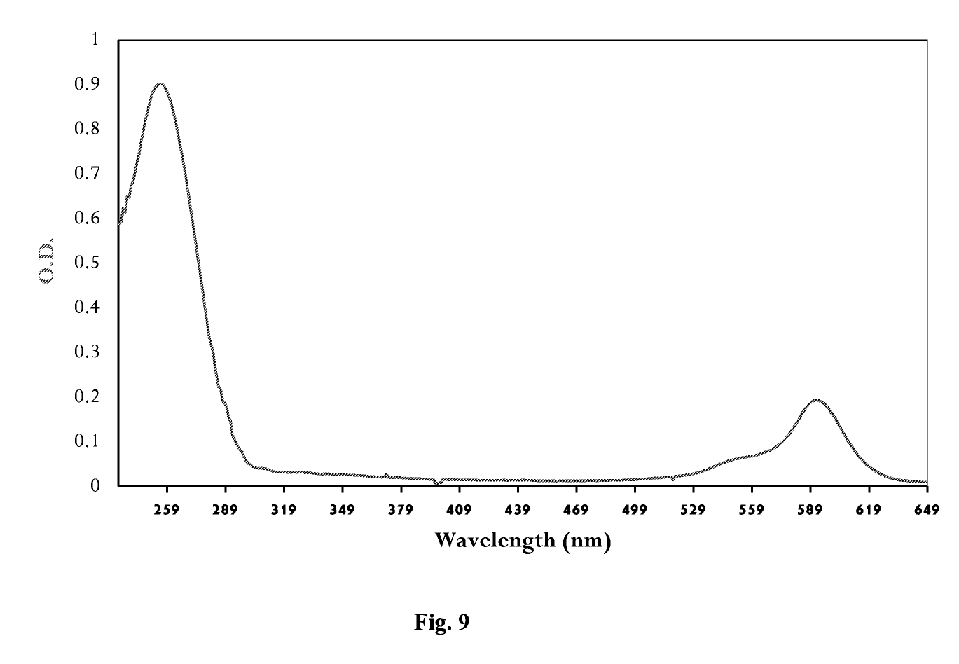

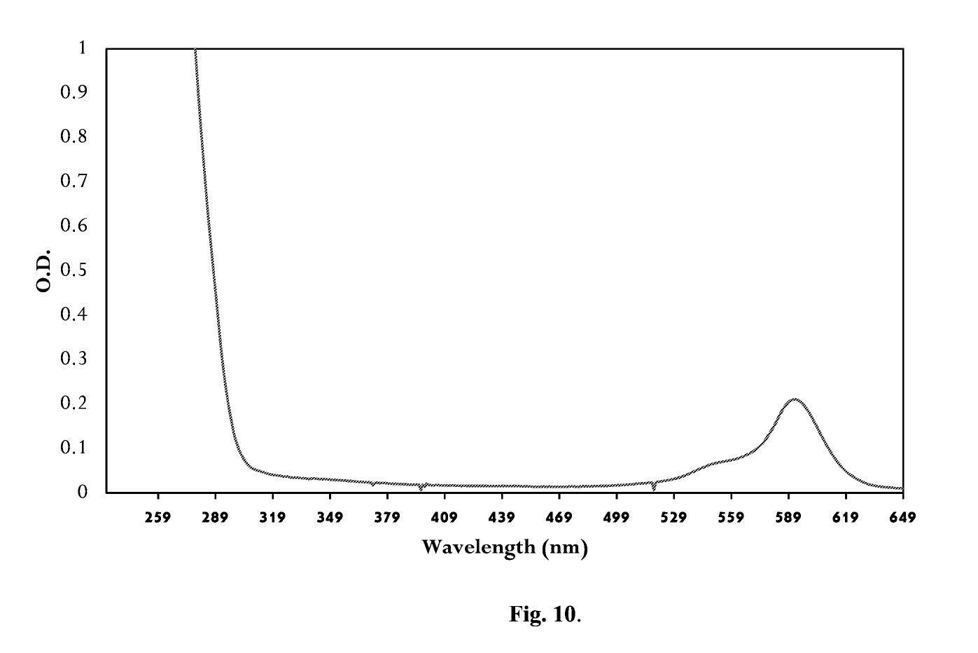

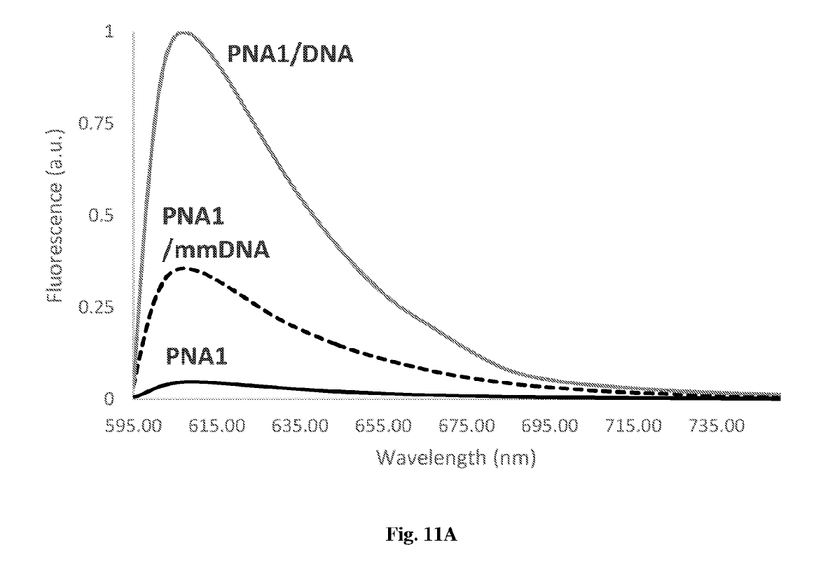

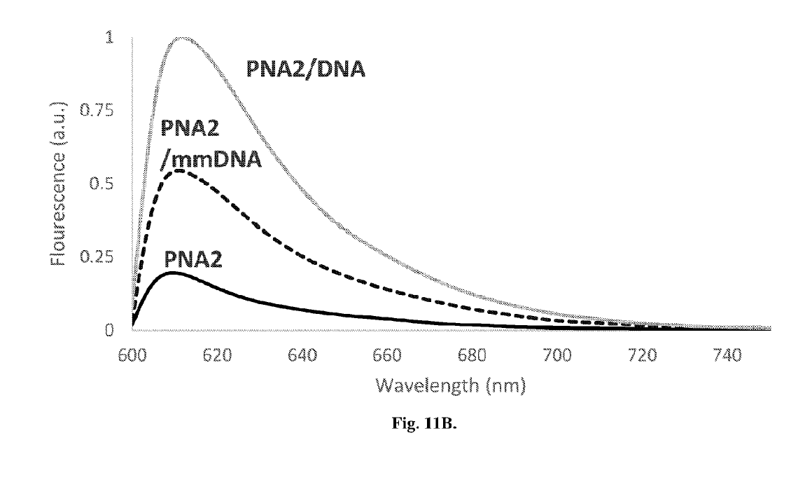

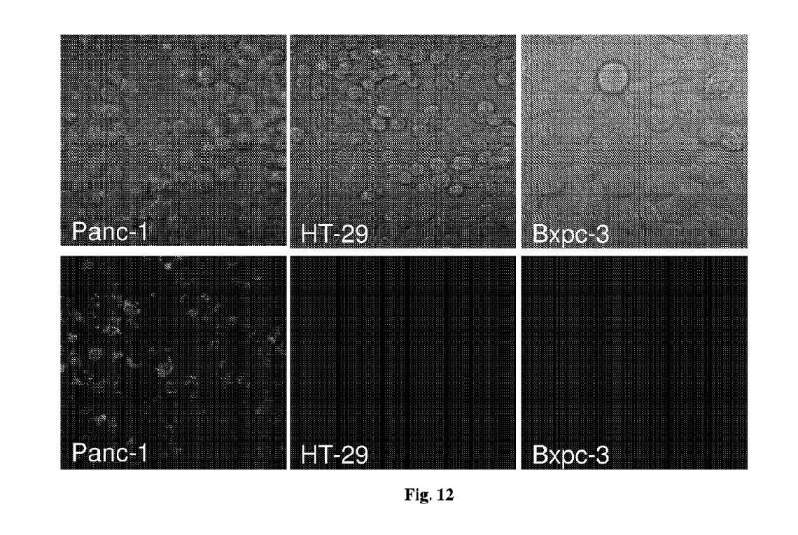

### DETAILED DESCRIPTION OF EMBODIMENTS

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

General Procedures and Materials

Manual solid-phase synthesis was performed by using 5 mL polyethylene syringe reactors (Phenomenex) that are equipped with a fritted disk. All column chromatography was performed using 60A, 0.04-0.063 mm Silica gel (Biolab, Israel) and manual glass columns. TLC was performed using Merck Silica Gel 60 F254 plates. HPLC purifications and analysis were performed on a Shimadzu LC-1090 system using a semi-preparative C18 reversed-phase column (Jupiter C18, 5u, 300 Å, 250×10 mm, Phenomenex) at 50° C. Eluents: A (0.1% TFA in water) and B (MeCN) were used in a linear gradient (11-40% B in 38 min) with a flow rate of 4 mL/min. NMR spectra were recorded on a 300 and 600 MHz Bruker NMR using deuterated solvents as internal standards. MS measurements of compounds 1-5 and BisQ were measured on a ThermoQuest Finnigan LCQ-Duo ESI mass spectrometer. Mass analysis of PNAs was acquired on an Orbitrap MS (Voyager DePro, Applied Biosystems, CA, USA). DNA oligos were purchased from Sigma-Aldrich, Israel. Dry DMF was purchased from Acros and Fmoc/Bhoc protected PNA monomers from PolyOrg Inc. (USA). The Fmoc-protected amino acids and reagents for solid phase synthesis were purchased from Merck (Germany).

### Example 1: Synthesis of BisQ

The synthesis of BisQ was performed as described in Scheme 2 below and according to the following procedure:

### 1-carboxymethyl-4-methylquinolinium Bromide (1)

Compound 1 was synthesized as previously described (L. Bethge, D. V. Jarikote and O. Seitz, Bioorg. Med. Chem., 2008, 16, 114-125) with some slight modifications: 4-methylquinoline (570 mg, 4 mmol) and bromoacetic acid (607 mg, 4.4 mmol) were suspended in 10 ml of dry toluene and refluxed for 24 h. The solvent was evaporated and the brown residue was dissolved in DCM and cooled to 0° C. Acetone (30 ml) was added dropwise, and the solid was collected by filtration and washed with acetone (3×10 ml). The crude solid was suspended in chloroform and stirred for 1 h. The solid was collected by filtration and washed with chloroform to afford 1 as a grey solid (825 mg, 80%). ¹H NMR (CD₃OD): 1.99 (3H, s, CH₃), 3.79 (2H, s, CH₂), 6.95 (2H, t, J=7.1, 2 ArH), 7.13 (1H, t, J=7.8, ArH), 7.22 (1H, d, J=8.9, ArH), 7.48 (1H, d, J=8.5, ArH), 8.15 (1H, d, J=6.1, ArH). MS: M_{obt}=202.20, M_{calc}=202.09

### 1-methyl-chloroquinolinium Iodide (2)

Compound 2 was synthesized as previously described (R. Lartia and U. Asseline, Chem.—Eur. J., 2006, 12, 2270-228) with some slight modifications: 4-chloroquinoline (1 g, 7 mmol) and iodomethane (4 ml, 45 mmol) were combined and heated to 45° C. for 4 h. The resulting solid was precipitated from a cold ether (60 ml) and vacuum dried to afford 2 as a yellow solid (1 g, 50%).

### 4-[[1-carboxymethyl-4(1H)-quinolinylidene]methyl]-1methyl quinoliniumbromide (3)

A mixture of 1 (560 mg, 2 mmol), 2 (600 mg, 2 mmol) and triethylamine (TEA, 4 mmol) in 6 ml dry DCM was stirred for one hour to produce a dark blue solution. As compound 3 decomposes rapidly, it was used in the next reaction without purification (see below).

Boc-Aeg-OtBu (4).

Boc/t-Bu protected PNA-backbone was synthesized as previously reported (Y. Kam, A. Rubinstein, A. Nissan, D. Halle and E. Yavin, Mol. Pharm., 2012, 9, 685-693).

Boc-Aeg(Dye)-OtBu (5).

To the stirring reaction mixture of Compound 3 from previous synthetic step, (2 mmol, 686 mg) equimolar amounts of PyBOP (1040 mg), PPTS (500 mg), NMM (220 ul) in 3 ml dry DMF were added. The mixture was stirred for 10 minutes following by the addition of 350 mg (1.3 mmol) of Boc-Aeg-OtBu. The reaction vessel was sealed and the reaction mixture was stirred under argon overnight at 45° C. The volatiles were removed under reduced pressure. The crude product was purified by silica gel column chromatography (0 to 15% MeOH gradient in DCM) to yield a blue colored paste (360 mg, 45%). ¹H NMR (CD₃C1): 8.3 (d, 2H, ArH), 7.8 (d, 1H, ArH), 7.67 (d, 2H, ArH), 7.53 (m, 4H, ArH), 7.34 (t, 2H, ArH), 7.0 (s, 1H, CH), 5.78 (d, 2H, CH₂), 4.38 (s, 1H, N—CH₂), 4.03 (s, 1H, N—CH₂), 3.96 (s, 3H, N⁺—CH₃), 3.9 (s, 2H, Gly-CH₂), 3.7 (t, 1H, N—CH₂), 3.28 (t, 1H, N—CH₂), 1.42 (s, 9H, t-Bu), 1.40 (s, 9H, t-Bu) MS: M_{obt}=599.36, M_{calc}=599.3

### Fmoc-Aeg(Dy)-OH (BisQ)

Compound 5 was dissolved in a 20 ml mixture of DCM/TFA (1:1). After two hours the solvents were evaporated and the resulting slurry was dissolved in 10 ml DCM. The pH was adjusted to ca. 10 by adding 10 equivalents (860 ul) of TEA. Next, (242 mg, 0.7 mmol) Fmoc-OSu were added dropwise under continuous stirring. After 12 h the solvent was evaporated and the crude mixture was purified by silica gel chromatography (20% MeOH in DCM) followed by further purification by preparative HPLC (Luna 10 microns, 100A, C-18 250×21.2 mm, Phenomenex), using an acetonitrile gradient (12-60% in 60 min.) in 0.1% TFA in H2O. Rₜ=42 min-Yield=40%. ¹H NMR (DMSO-d6): 8.73 (d, 1H, ArH), 8.63 (d, 1H, ArH), 8.32 (m, 1H, ArH), 7.97-7.87 (m, 5H, ArH), 7.71 (m, 5H, ArH), 7.56 (m, 3H, ArH), 7.41 (t, 2H, ArH), 7.31 (t, 2H, ArH), 7.26 (s, 1H, CH), 5.55 (s, 1H, CH₂), 5.33 (s, 1H, CH₂), 4.37 (m, 1H, Fmoc-CH₂), 4.35 (s, 0.5H, Gly-CH₂), 4.30 (d, 1H, Fmoc-CH₂), 4.24 (t, 0.5H, Fmoc-CH), 4.20 (t, 0.5H, Fmoc-CH), 4.12 (s, 3H, N⁺—CH₃), 4.02 (s, 1.4H, Gly-CH₂), 3.60 (t, 1H, N—CH₂), 3.39 (m, 2H, N—CH₂), 3.14 (m, 1H, N—CH₂). ¹³C-NMR (DMSO-d6): (two rotamers) δ ppm: 37.7, 38.6 (N—CH₂), 41.8 (CH₃), 46.5 (Fmoc-CH), 46.8, 47 (2×N—CH₂), 47.6, 49 (2×Gly-CH₂), 53.6, 54.2 (2×CH₂), 65.2, 65.4 (2×Fmoc-CH₂), 96.6, 96.5 (2×ArC), 107.4, 109.3, 109.5 (3×ArC), 115.4, 113 (2×ArCq), 117, 117.7 (2×ArC), 119.9 (Fmoc-ArC), 120.1 (ArCq), 124.4 (ArCq), 124.9 (Fmoc-ArC), 125.1 (ArCq), 125.4, 125.6 (2×ArC), 126.3, 126.8, 127.4 (3×Fmoc-ArC), 132, 132.7 (2×ArC), 138.4, 140.5 (2×Fmoc-ArCq), 142.9, 143.1, 143.9 (3×ArC), 144 (ArCq), 148, 149.7 (2×Fmoc-ArCq), 155.9, 156.3 (2×Fmoc-COONH), 166.1, 166.5 (2×Dye-CON), 170, 171.9 (Gly-COOH). HRMS: M_{obt}=665.275, M_{calc}=665.275.

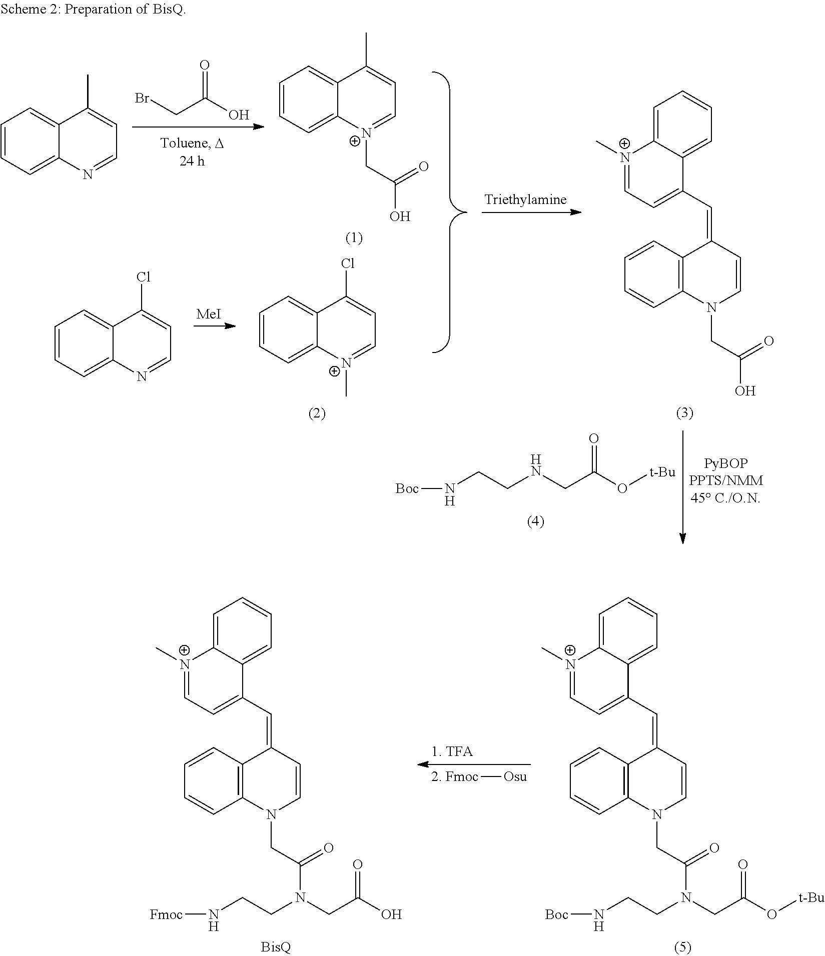

### Example 2: Solid Phase Synthesis of PNA1 and PNA2

Coupling of First Amino onto Novasyn TGA Resin.

The resin (250 mg, 0.2 mmol/g) was allowed to swell in 10 ml DMF for 30 min. For pre-activation, DIC (5 eq.) and DIMAP (0.1 eq.) were added to a solution of Fmoc-protected glycine (10 eq.) in DCM (15 ml) in an ice bath. After 15 min, the mixture was evaporated, re-dissolved in dry DMF and added to the resin. After 2.5 h, the resin was washed with DMF (5×2 mL), CH₂C12 (5×2 mL) and the procedure was repeated.

Fmoc Cleavage.

A solution of DMF/piperidine (4:1, 1 ml) was added to the resin. After 2 min the procedure was repeated. Finally the resin was washed with DMF (3×1 ml), DCM (3×1 ml).

Coupling of Fmoc-Bhoc-PNA-Monomers.

4 eq. of PNA monomer, 4 eq. HATU, 4 eq. HOBt and Seq. of dry DIPEA in DMF (1.5 ml) were mixed in a glass vial equipped with a screw cap. After 3 min of pre-activation, the solution was transferred to the resin. After 60 min, the reaction mixture was discarded and the resin was washed with DMF (2×1 ml) and DCM (2×1 ml).

Coupling of Fmoc-Aeg(Dye)-OH (BisQ).

4 eq. of PNA monomer, 4 eq. HATU, 4 eq. HOBt and Seq. of dry DIPEA in DMF (1.5 ml) were mixed in a glass vial equipped with screw cap. Following 3 min of pre-activation, the solution was transferred to the resin. After 60 min, the procedure was repeated and finally the resin was washed with DMF (2×1 ml) and DCM (2×1 ml).

Cleavage of PNA from Resin.

1 ml TFA was added to the dry resin. After 2 h another portion of TFA was added. The combined TFA solutions were concentrated in vacuo.

TABLE 1
PNA and DNA sequences.

| Name | Description | Construct |
|---|---|---|
| PNA-1 | BisQ paired with G | (D-Lys)₄-CCTCGA[BisQ]TACCGCATCC-NH₂ |
| PNA-2 | BisQ paired with T | (D-Lys)₄-CCTCGACT[BisQ]CCGCATCC-NH₂ |
| DNA | Mismatch in adjacent | 5′-GTAGTTGGAGCTG [symbol] TGGCGTAGGCAAGAGT |
| | nucleotide | |
| Mutated | full complementarity | 5′-GTAGTTGGAGCTG [symbol] TGGCGTAGGCAAGAGT |
| DNA | | |

Underline denotes sequence complementary to PNA.
Double underline denotes mutation site in KRAS sequence.

PNA Purification.

PNAs were precipitated from the concentrated TFA solution by addition of cold diethyl ether (15 ml). The precipitate was collected by centrifugation and decantation of the supernatant. The residue was dissolved in water and purified by semi preparative HPLC. The purified PNAs were analysed by Orbitrap-MS.

Fluorescence Spectrometry.

Fluorescence spectra were recorded by using a Jasco FT-6500 spectrometer. Measurements were carried out in fluorescence quartz cuvettes (10 mm) at 0.5-1.5 μM concentration in a PBS buffered solution (100 mM NaCl, 10 mM NaH₂PO₄, pH 7 Quantum yields were determined relative to fluorescein in PBS as described (doi:10.1016/j.tet.2013.03.005). PNAs were hybridized to complementary DNA by heating a 1:1 mixture of PNA:DNA (10-30 μM) to 95° C. for 5 min followed by slow cooling to 25° C. Samples were excited at 587 nm and emission spectra were recorded at 600-800 nm.

TABLE 2
Photophysical properties of BisQ and PNAs.

| | λₘₐₓabs | εₘₐₓ | λₘₐₓem | |
|---|---|---|---|---|
| Compound | (nm) | [M⁻¹cm⁻¹] | (nm) | φ |
| BisQ | 591 | 41,370 | n.a. | n.a. |
| TO | 505 | 43,000 | n.a. | n.a. |
| PNA1 | 588 | 83,000 | n.a. | n.a. |
| PNA1 + DNA | 587 | 112,30 | 609 | 0.22 |
| PNA2 | 593 | 82,219 | n.a. | n.a. |
| PNA2 + DNA | 593 | 94,535 | 610 | 0.25 |

### Example 3: Cell Experiments

Cell Lines and Culture.

Three cell lines were used: Panc-1, BxPC-3 (human pancreatic carcinoma, epithelial-like), and HT-29 (human colon adenocarcinoma grade II). Cell lines were purchased from the American Type Culture Collection (ATCC, Manassas, Va., USA). Panc-1 and HT-29 expressing mutated and wild type KRAS, respectively, were cultured (37° C., 5% CO₂), in DMEM medium and supplemented with 10% fetal calf serum, 2 mM L-glutamine, and 0.1 mg/mL Streptomycin (Beit Haemek Biological Industries, Israel). BxPC-3 cells expressing wild type KRAS were cultured in RPMI 1640 medium supplemented with 10% fetal calf serum, 2 mM L-glutamine, and 0.1 mg/mL Streptomycin.

Cellular Uptake Analysis.

Twenty four hours prior to PNA addition, Panc-1, HT-29, and BxPC-3 were plated separately on chamber slides (Ibidi GmbH, Munich, Germany) until reaching 70-80% confluence.

Hybridization and Imaging in Living Cells.

Before adding the PNAs, the medium was replaced and the cells were incubated (37° C., humidified atmosphere containing 5% CO₂) with 0.5 μM of PNA1 and PNA2 in complete medium. Cells were washed with PBS (×3) prior to cell imaging and the intracellular fluorescence was measured after 3 hours by confocal microscopy. Design and synthesis of a new surrogate base (BisQ) with the unique feature of far-red emission. BisQ was introduced into PNAs that targets the mutated kRAS oncogene. A PNA probe with a short cell penetrating peptide (CPP) consisting of 4 D-Lysines was shown to readily penetrate living cancer cells and fluoresce in the far-red region (λmax=609 nm) exclusively in pancreatic cancer cells (Panc-1) that express the mutated form of kRAS but not in pancreatic cancer cells that are non-mutated (wild type) in kRAS (BxPC-3).

### Example 4: Synthesis of Acr-2

The synthesis of Acr-2 was performed as described in Scheme 3 below and according to the following procedure:

Preparation of Benzyl Glycolate (1a).

Glycolic acid (3 g) and DBU (6 g, 1 eq) were added to 80 ml of toluene and allowed to stir for 15 min. Benzyl bromide (8 g or 5.6 mL, 1.2 eq) was added drop wise and the reaction mixture was refluxed for 6 h. The reaction mixture was then extracted with 1M HCl (50 mL×2) and water (50 ml).The organic layer was dried over anhydrous Na₂SO₄ and concentrated in vacuum. The concentrate was chromatographed (silica, 15% EtOAc in hexane) to yield a colourless liquid. 1H NMR (CDCl3): δ 7.36 (s, 5H), 5.21 (s, 2H), 4.20 (d, 2H), 2.93 (t, 1H).

Preparation of Triflate of Benzyl Glycolate (1b).

To a solution of benzyl glycolate (2 g) and pyridine 91.05 g, 1.1.eq) in DCM (50 ml) at −200 C was added triflic anhydride (3.73 g, 1.1 eq) over a period of 5-10 min. After complete addition, the reaction mixture was stirred for 30 min and warmed to RT, and stirred for additional 30 min. The reaction mixture was evaporated and rapidly passed through the short column of silica gel eluting with DCM. The fractions were evaporated to pale yellow oil which solidified when stored at 00 C. 1H NMR (CDCl3): δ 7.38 (s, 5H), 5.28 (s, 2H), 4.93 (s, 2H)

Synthesis of Acridinium Ester (1c).

To a solution of 9-methylacridine (200 mg) in dry DCM (5 ml) at −150 C, was added triflate of benzyl glycolate (340 mg, 1.1 eq) over a period of 5-10 min. After complete addition, the reaction mixture was stirred for 30 min and warmed to RT, and stirred overnight. Dry diethyl ether was added during which the product precipitated as a solid and subsequently filtered and washed with ether. 1H NMR (300 MHz, acetone): δ 8.80 (d, 2H) 8.46 (d, 2H), 8.32 (t, 2H), 7.98 (t, 2H), 7.38 (s, 5H), 5.32 (s, 2H), 3.70 (s, 2H), 3.55, (s, 3H)

Synthesis of Acridinium Ester Dye (1d).

9 methyl acridinium ester (163 mg) was suspended id dry DCM (5 ml) and allowed to stir for 5 min. 1-methyl-chloroquinolinium iodide was added and stirred for 5 min. Triethylamine was added in one portion to the reaction mixture (solution turned to red to purple) and allowed to stir overnight. Solvent was evaporated and the crude product was washed repeatedly with ether. The dark color residue was purified by column chromatography (10% MeOH in DCM). 1H NMR (300 MHz, acetone): δ 9.11 (d, 1H), 8.89 (d, 1H), 8.48 (d, 1H), 8.26 (t, 1H), 8.00 (t, 1H), 7.89 (d, 2H), 7.65, (d, 1H) 7.50 (t, 4H), 7.38, (t, 5H) 7.08, (t, 2H) 7.05, 6.01, (d, 1H) 5.32 (s, 2H), 5.25 (s, 2H), 4.68 (s, 3H), 3.88, (s, 1H). 13C NMR (75 MHz, acetone): δ 168.70, (C═O), 157.57 (Ar—C), 146.89 (Ar—C), 142.59 (Ar—C), 140.21 (Ar—C), 135.92 (Ar—C), 135.13 (Ar—C), 131.00 (Ar—C), 128.93 (Ar—C), 128.48 (Ar—C), 127.57 (Ar—C), 127.02 (Ar—C), 122.09 (Ar—C), 119.00 (Ar—C), 118.79 (Ar—C), 114.88 (Ar—C), 111.96 (Ar—C), 66.93, (Ar—CH₂), 48.93 (N—CH₂), 44.47 (CH₃). HRMS: Mobs=483.206, Mcalc=483.207

Synthesis of Benzyl Glycolate De Protected Acridinium Ester Dye (1e).

The acridinium ester (100 mg) protected by a benzyl group was suspended in a 30% solution of HBr in acetic acid (3 mL) and heated for 30 min at 500 C, and the solvent was evaporated in vacuum. The residue was co evaporated with toluene (5 ml) and washed thoroughly with diethyl ether and was purified by a DCM-water extraction (30 ml×3). The acid was immediately coupled to the PNA backbone without further purification. 1H NMR (300 MHz, D20): δ 8.77 (d, 1H), 8.56 (d, 1H), 8.30 (t, 5H), 8.21 (t, 3H), 8.10 (t, 1H), 7.69 (t, 2H), 6.69 (d, 1H), 5.96 (s, 2H), 4.37 (s, 3H), 4.27 (s, 1H).

Synthesis of Acridine-Quinoline Dye Attached to Fmoc Backbone (1f).

To a solution of Acr-acid (100 mg) in dry DMF (3 ml) at 00 C, PyBOP (1.28 eq), HOBT (1.28 eq) and NMM ((1.28 eq) were added and stirred under argon for 15 min until a clear solution was obtained. To this solution, a separately prepared sample of Fmoc-Aeg-allyl ester (1.28 eq) and NMM ((1.28 eq) in DMF (1 ml) was added and the reaction mixture was allowed to stir for 12 h at RT. Upon completion of the reaction (as indicated by tlc), the reaction mixture was diluted with water prior to extraction with EtOAc (3×20 mL). The combined organic layers were washed with 10% NaHCO3 followed by washing with 10% citric acid. The combined organic layers were washed again with aqueous 10% NaHCO3 followed by water and brine. The organic layer was dried with anhydrous Na2SO4 and concentrated under vacuum. The crude product was purified by column chromatography using 10% MeOH in DCM as eluent. ¹H NMR (300 MHz, CDCl3): δ 8.53 (d, 1H), 8.46 (d, 1H), 8.42 (d, 1H), 8.13 (t, 1H), 8.03 (d, 2H), 8.00 (d, 1H), 7.94 (t, 1H), 7.90 (d, 2H), 7.79 (d, 1H), 7.71 (d, 2H), 7.66 (d, 1H), 7.49 (t, 2H), 7.43 (t, 2H), 7.36 (t, 2H), 7.10 (t, 2H), 7.05 (t, 2H), 7.01 (t, 2H), 6.98 (t, 1H), 6.02 (q, 1H), 5.47 (d, 1H), 4.93 (d, 2H), 4.35 (s, 3H), 4.15 (d, 1H), 3.89 (d, 2H), 3.74 (s, 2H), 3.47 (d, 2H), 2.95 (s, 2H), 2.87 (s, 2H).

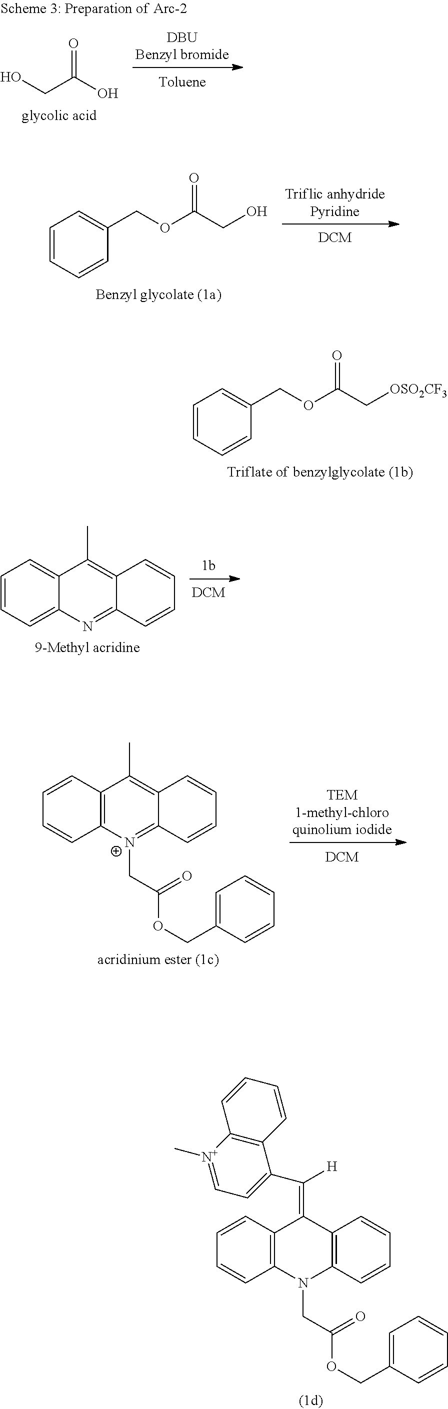

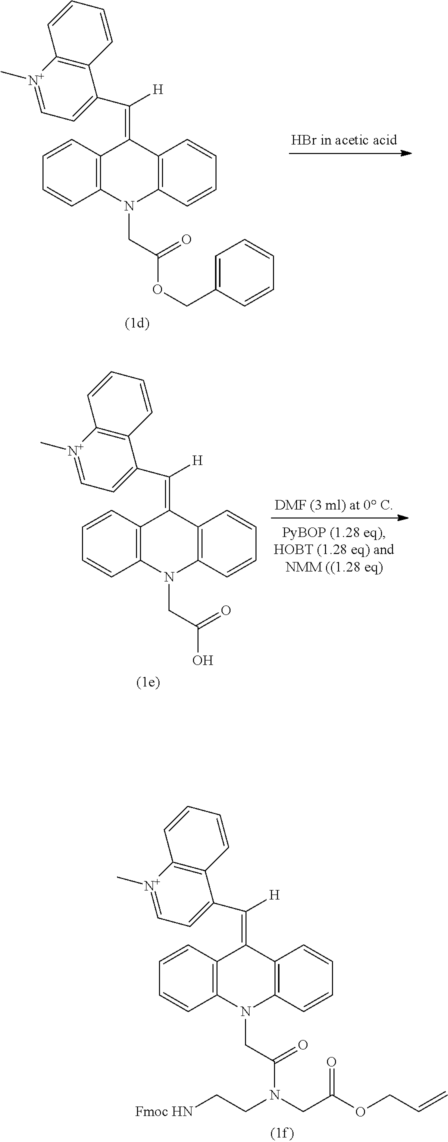

### Example 5: The Synthesis of Acr-1

The synthesis was performed in a similar manner to that of Acr-2, as described in scheme 4 below.

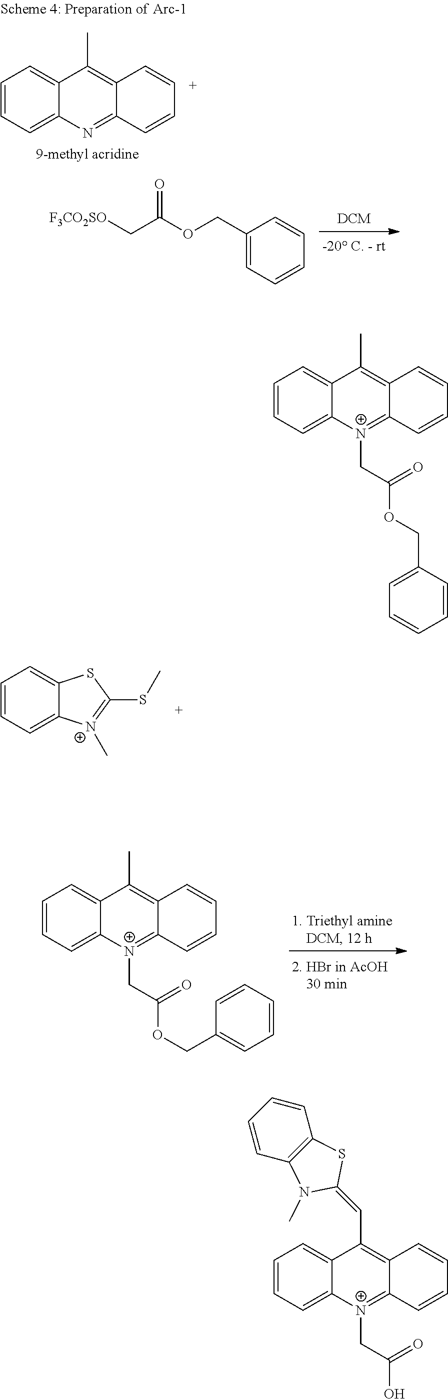

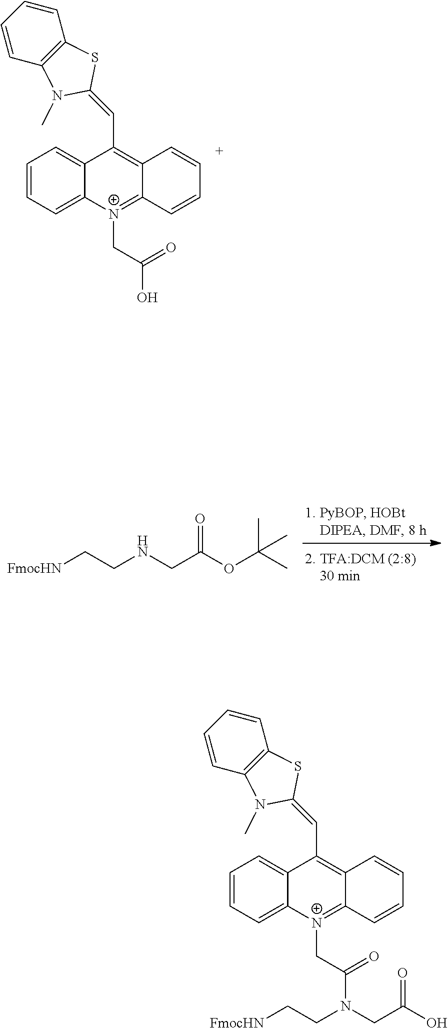

1.-27. (canceled)

28. A conjugate comprising at least one red to near infra-red (NIR) emitting probe component connected as surrogate base into at least one complementary component, said red to far-red emitting probe component comprises at least one methine bond.

29. A conjugate according to claim 28, wherein said red to far-red emitting probe component is emitting radiation in the range of 600 nm to 790 nm.

30. A conjugate according to claim 28, wherein said red to NIR emitting probe component is selected from the group consisting of:

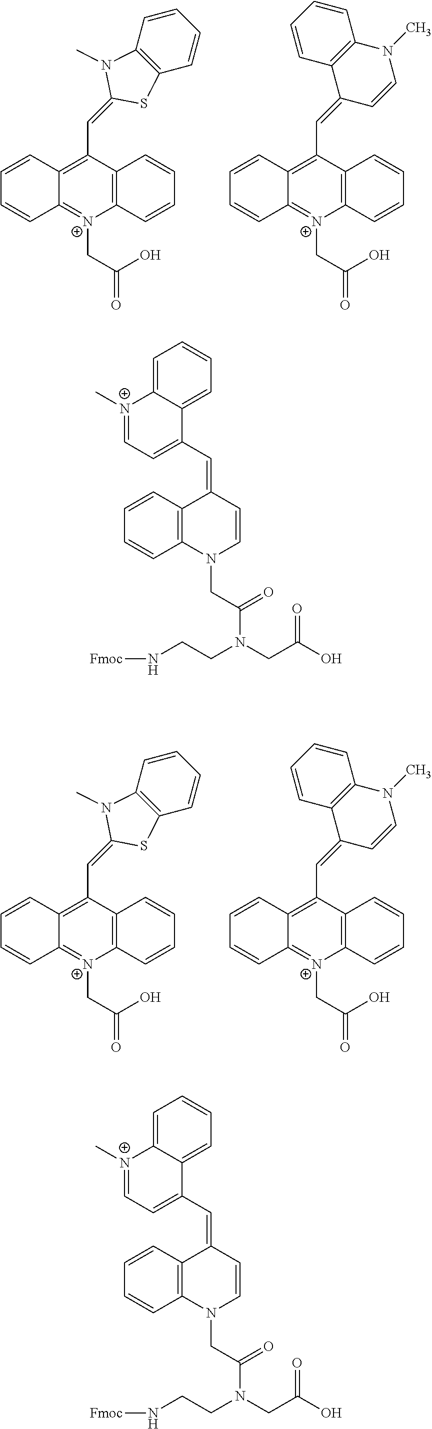

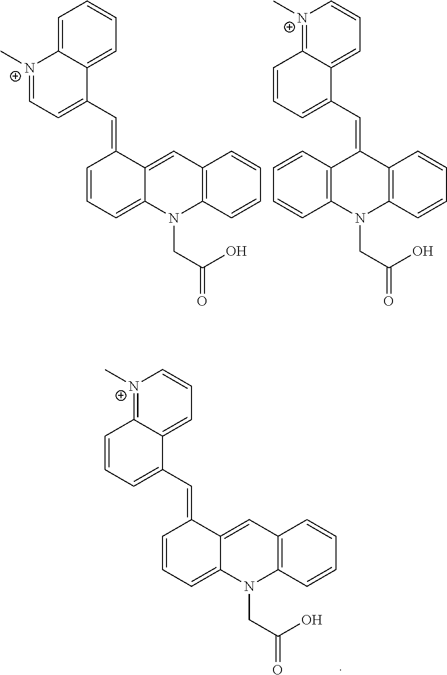 [SMILES: C[n+]1ccc(/C=c2\cccc3c2=Cc2ccccc2N3CC(=O)O)c2ccccc21 ; C[n+]1cccc2c(C=C3c4ccccc4N(CC(=O)O)c4ccccc43)cccc21 ; C[n+]1cccc2c(/C=c3\cccc4c3=Cc3ccccc3N4CC(=O)O)cccc21]

31. A conjugate according to claim 28, wherein said at least one complementary component is selected from the group consisting of a Peptide nucleic acid (PNA), a DNA sequence and an RNA sequence and any combinations thereof.

32. A conjugate according to claim 28, wherein said at least one complementary component comprises an oligonucleotide sequence complementary to a targeted gene sequence, said gene sequence being optionally a DNA or RNA sequence or a mutation sequence associated with a disorder, a condition or a disease.

33. A composition comprising at least one conjugate according to claim 28.

34. A method of detecting a genetic condition in a fetus comprising the steps of incubating a conjugate according to claim 28, with a sample of fetus living cells; and exposing said incubated cells to red-NIR fluorescence detector at the red to far red spectrum, thereby diagnosing a genetic condition of said fetus.

35. A method of in vitro diagnosis of a malignant condition in a tissue of a subject comprising the steps of incubating a conjugate according to claim 28, with a tissue excised from said subject, exposing said incubated tissue to red-NIR fluorescence detector, thereby diagnosing malignancy in said tissue.

36. A method of in vivo diagnosis of a malignant condition or disorder comprising the steps of administering to a subject a conjugate according to claim 28, imaging at least a part of said subject's body using red-NIR fluorescence detector, thereby diagnosing malignant disorder in said subject.

37. A method of determining the extent of removal of malignant tissue during or after a malignancy removal surgery using fluorescence guided surgery comprising the steps of removing a malignant tumor from a subject's body, incubating a conjugate as defined in claim 28, with at least a part of the boarders of said removed malignant tissue, exposing said incubated tissue to red-NIR fluorescence detector, thereby determining the extent of removal of malignant tissue.

38. A method for early diagnosis of a malignant disorder in a subject at risk comprising the steps of administering to a subject a conjugate according to claim 28, imaging at least a part of said subject's body using a red-NIR fluorescence detector, thereby diagnosing malignant disorder of said subject.

39. A kit comprising a conjugate as defined in claim 28, for use in the diagnosis of a genetic condition, disease or disorder, including instructions for use thereof.

40. A kit comprising a conjugate as defined in claim 28, for use in the diagnosis of a malignant condition, disease or disorder, including instructions for use thereof.
